(19) United States
(12) Patent Application Publication
(10) Pub. No.: US 2024/0173502 A1
(43) Pub. Date: May 30, 2024

(54) DEVICE AND METHOD FOR PNEUMATICALLY OR HYDRAULICALLY SHAPABLE STYLET FOR AN ENDOTRACHEAL BREATHING TUBE

(71) Applicant: Arthur Cronin, Hummelstown, PA (US)

(72) Inventor: Arthur Cronin, Hummelstown, PA (US)

(21) Appl. No.: 18/520,045

(22) Filed: Nov. 27, 2023

Related U.S. Application Data
(60) Provisional application No. 63/385,294, filed on Nov. 29, 2022.

(51) Int. Cl. A61M 16/04; A61B 1/267

(52) U.S. Cl. CPC A61M 16/0488; A61B 1/267; A61M 16/0418; A61M 16/0463; A61M 16/0472

(57) ABSTRACT

A system may include a device for positioning an endotracheal breathing tube including a compressible handpiece for producing fluid pressure, wherein the fluid pressure is produced in a graded manner based on an operator compressing the compressible handpiece, and a stylet linearly extending from a first end of the stylet to a tip of the stylet, the first end of the stylet coupled to the compressible handpiece, the stylet configured to be inserted into a lumen of an endotracheal breathing tube and deflect into a curved shape when the compressible handpiece is compressed to produce fluid pressure within the stylet, wherein the stylet has a length substantially equal to a length of the endotracheal breathing tube.

### BACKGROUND

### 1. Field

Embodiments generally relate to systems and methods for guiding placement and/or positioning of endotracheal breathing tubes in patients. Embodiments may relate to systems and methods including a medical device for aiding placement of an endotracheal breathing tube using handheld and hand-operated pneumatic or hydraulic controls to guide placement and/or positioning.

### 2. Technical Considerations

When placing an endotracheal breathing tube in a patient, medical practitioners face various challenges. In some instances, medical practitioners may have difficulty visualizing a target area for placing an endotracheal breathing tube within a patient. A target area may include a gap between the vocal cords in which an endotracheal breathing tube is to be positioned. In some instances, medical practitioners may have difficulty directing the endotracheal breathing tube between vocal cords and about an inch further into a trachea of a patient for intubation. The various issues faced by medical practitioners when placing an endotracheal breathing tube in a patient may result in unnecessary errors and multiple attempts of placement until successful placement of the endotracheal breathing tube is achieved. Multiple attempts of placement of an endotracheal tube may be harmful to the patient, especially in emergency scenarios. Similarly, errors when placing an endotracheal breathing tube in a patient may be costly and may harm the patient.

In some instances, stylets may be used to help with guidance and placement of an endotracheal breathing tube. Current stylets are typically made of metal, and often are malleable. Current stylets are placed inside an endotracheal breathing tube like lead in a pencil, and the endotracheal breathing tube with the stylet inside it may be bent into a gentle curve or hockey stick shape. Current stylets are sometimes not sufficiently rigid to retain the required angulation for some patients, and some current stylets may be too rigid resulting in an incorrect shape for some patients. Such current stylets do not allow for use on different patients and do not allow for ease of use for medical practitioners to achieve successful intubation.

Any delay in the intubation process, either due to difficulty visualizing the vocal cords or difficulty directing the tip of the endotracheal breathing tube between the vocal cords, increases risk of complications. These complications may include regurgitation and aspiration of stomach contents, trauma to the airway with bleeding and tissue swelling, and possibly failure to intubate. Failure to intubate may lead to attempted mask ventilation and/or a front of neck surgical airway which may result in possible hypoxic death.

Some patient attributes and/or conditions may also make use of current stylets difficult for medical practitioners to achieve successful intubation. For example, a patient may have congenital anomalies such as micrognathia or a very large tongue, post radiation cervical fibrosis, an airway tumor or infection and soft tissue swelling as with a tonsillar abscess. Such conditions may limit mouth opening of a patient and make laryngoscopy and endotracheal intubation challenging. Trauma to the airway or an unstable cervical spine fracture may risk cervical spinal cord injury with laryngoscopy, which can also necessitate a more challenging intubation technique which does not displace the cervical tissue.

Additionally, successful endotracheal intubation on a first attempt is even less likely for less experienced medical practitioners or in emergency scenarios. Thus, there may exist a higher risk of patient complications. In some instances, least trained medical providers are required to perform endotracheal intubation in the least favorable and most stressful conditions with only basic equipment where duration of laryngoscopy and number of attempts can impact the patient's health outcome. Endotracheal intubation remains difficult as current tools and procedures have proven inadequate for many medical practitioners.

### SUMMARY

Embodiments may relate to a device including a stylet. The stylet may include a distal end and a proximal end. The stylet may be positioned in an endotracheal tube. The device may include a handpiece removably coupled to the proximal end of the stylet. The handpiece may be compressible and may be configured to transmit fluid pressure to an internal portion of the stylet. The stylet may be configured to change shape upon transmission of fluid pressure from the handpiece.

In some embodiments, the device may include a camera positioned at the distal end of the stylet.

In some embodiments, the stylet may be configured to deflect (e.g., to form a curved shape) upon transmission of fluid pressure from the handpiece.

Embodiments may relate to a device for positioning an endotracheal breathing tube. The device may include a compressible handpiece for producing fluid pressure. The fluid pressure may produced in a graded manner based on an operator (e.g., a medical practitioner) compressing the compressible handpiece. The device may include a stylet linearly extending from a first end of the stylet to a tip of the stylet. The first end of the stylet may be coupled to the compressible handpiece. The stylet may be configured to be inserted into a lumen of an endotracheal breathing tube. The stylet may deflect into a curved shape when the compressible handpiece is compressed to produce fluid pressure within the stylet.

In some embodiments, the stylet may be configured to maintain a linear shape and return to the linear shape when the compressible handpiece is not compressed and no fluid pressure is produced within the stylet.

In some embodiments, the device may include a first continuous channel arranged within an interior of the stylet extending from the first end of the stylet to the tip of the stylet. The first continuous channel may include a plurality of airtight pockets including apertures for fluid to enter the airtight pockets. The airtight pockets may be distributed along the first continuous channel (e.g., along an external wall of the first continuous channel) of the stylet and configured to inflate when the compressible handpiece is compressed to produce fluid pressure within the first continuous channel of the stylet and thereby cause fluid to enter the airtight pockets through the apertures of the airtight pockets.

In some embodiments, the airtight pockets may be attached to a wall (e.g., an external wall) of the stylet. The airtight pockets may be configured to cause the stylet to deflect when at least one of the airtight pockets are inflated.

In some embodiments, the device may be in combination with an endotracheal breathing tube. The endotracheal breathing tube may include a lumen. The stylet may be inserted into the lumen of the endotracheal breathing tube. The stylet may have a length substantially equal to the length of the endotracheal breathing tube. In some embodiments, when the compressible handpiece is compressed to produce fluid pressure, the fluid pressure may cause the stylet to form a graded curve, each in the same direction, thereby causing the endotracheal breathing tube to form a curve based on the stylet deflecting into a curved shape when the compressible handpiece is compressed to produce fluid pressure within the stylet.

In some embodiments, the airtight pockets may be arranged along the continuous channel (e.g., along an external wall of the continuous channel) such that the stylet is configured to deflect and thereby cause an endotracheal breathing tube to achieve a curved shape in conformance with the stylet, the airtight pockets arranged along the continuous channel according to any one or more of: a degree of angulation induced at each pocket, a number and location of the airtight pockets used to create the degree of angulation, and/or a sequence of inflation of the airtight pockets and the degree of angulation as the compression is increased on the compressible handpiece to exceed an opening pressure.

In some embodiments, the compressible handpiece may be configured to generate immediate tactile and/or visual feedback based on a relationship between compression of the compressible handpiece and a degree of curvature of the stylet.

In some embodiments, the device may further include a fiber extending from a convex surface of the tip of the stylet to a tension control unit attached to the compressible handpiece. The fiber may be coupled with the tension control unit. The fiber and tension control unit (e.g., in combination) may be configured to permit deflection of the tip of the stylet in the convex direction when tension is applied to said fiber (e.g., via the tension control unit).

In some embodiments, the device may be in combination with a control unit and at least one electrical power source. The device may also be in combination with a light emitting diode (LED) and a video camera electrically coupled to the at least one electrical power source. The LED and the video camera may be arranged internal to the stylet at the tip of the stylet. The device may also be in combination with a display device electrically coupled to the at least one electrical power source.

In some embodiments, the compressible handpiece and the stylet may be independent components (e.g., separate components) that are configured to be coupled and decoupled to each other while preserving any one or more of electrical function, fluid pressure function, concave tip deflection, convex tip deflection, medication delivery, and/or suction function.

In some embodiments, the device may further include a second continuous channel arranged within the interior of the stylet extending from the first end of the stylet to the tip of the stylet. The second continuous channel may be configured to deliver medication from the compressible handpiece at the first end of the stylet to the tip of the stylet. When a control button for the medication release in the handpiece is depressed, the medication may exit the tip of the stylet as a spray.

In some embodiments, the device may further include a hollow continuous channel arranged within the interior of the stylet extending from the first end of the stylet to the tip of the stylet. The hollow continuous channel may be configured to receive a guidewire placed percutaneously from below vocal cords of a patient for retrograde endotracheal intubation and for suction of material from an area at the tip of the stylet.

In some embodiments, the device may further include a first tubular connector attached to the compressible handpiece. The first tubular connector may be configured to couple with a tubular connector on an external end of an endotracheal breathing tube to provide for movement of the endotracheal breathing tube by moving the compressible handpiece.

Embodiments may relate to methods for positioning an endotracheal breathing tube. A method may include inserting a stylet into a lumen of an endotracheal breathing tube. The method may include compressing a compressible handpiece coupled to the stylet. Compressing the compressible handpiece may cause fluid pressure to increase in a first channel within the stylet, thereby causing the stylet to deflect and the endotracheal breathing tube to deflect in conformance with the stylet. The method may include positioning the endotracheal breathing tube in a patient by moving the stylet via the compressible handpiece. The method may include removing the stylet from the endotracheal breathing tube once the endotracheal breathing tube is positioned in the patient.

In some embodiments, positioning the endotracheal breathing tube may include positioning the endotracheal breathing tube in a trachea of the patient.

In some embodiments, positioning the endotracheal breathing tube may include positioning the endotracheal breathing tube in a posterior pharynx of the patient.

In some embodiments, the method may further include inserting a needle into a front of a neck of the patient such that the needle is inserted into a trachea of the patient. The method may include inserting a guidewire blindly through the needle such that the guidewire advances in a cephalad direction through vocal cords of the patient. The method may include producing, with a video camera attached to the stylet and a display device, a visual display of the guidewire in the patient. The method may include retrieving, with a suction channel of the stylet, the guidewire such that the guidewire is advanced up the suction channel of the stylet where the guidewire exits the compressible handpiece. The method may include advancing the endotracheal breathing tube into the trachea of the patient using the guidewire.

Embodiments may relate to a device that may facilitate positioning of an endotracheal breathing tube including a compressible handpiece that develops pneumatic or hydraulic pressure in a graded fashion created by the user increasing compression strength of their grip. The device may include a guidance stylet that extends from the handpiece and may be inserted down the lumen of the endotracheal breathing tube, the guidance stylet having a length similar to the length of the endotracheal breathing tube.

In some embodiments, the guidance stylet may maintain a linear shape and may return to this linear shape unless a curve is introduced by application of pneumatic or hydraulic pressure into the pockets or deflection by application of tension to the fiber which runs from the handpiece control to a tip of the guidance stylet.

In some embodiments, the guidance stylet may have a channel continuous from the handpiece to a plurality of apertures to airtight and fluid-tight pockets distributed over the internal several inches of the guidance stylet that may inflate when the pneumatic or hydraulic pressure from the handpiece is transmitted by fluid or air down the channel in the guidance stylet and is applied to inflate the said pockets.

In some embodiments, the graded pneumatic or hydraulic pressure and inflation of the said pockets may cause a graded curve, each in the same direction, in the guidance stylet and may induce a curve in the overlying endotracheal breathing tube.

In some embodiments, the said pockets may be positioned to achieve a specific shape in the guidance stylet and overlying endotracheal breathing tube, including 1) the degree of angulation induced at each pocket, 2) the number and the locations of the pockets recruited to create the angulation, and 3) the sequence of said pocket inflation and angulation as the grip compression is increased over its opening pressure.

In some embodiments, the device may provide palpable and immediate tactile and visual feedback informing the relationship between grip compression strength and the degree of curvature of the endotracheal breathing tube and guidance stylet.

In some embodiments, the device may include a fiber running from the convex surface of the distal end of the guidance stylet up to a tension control unit on the handpiece which may permit deflection of the tip of the stylet in the convex direction when tension is applied to said fiber.

In some embodiments, the device may include a control unit, electrical power source, wires running from the power source to the internal tip of the guidance stylet to a LED and video camera, and back to a video screen for viewing and possible transmission for remote viewing.

In some embodiments, the handpiece and the guidance stylet may be independent components but can be interlocked with preservation of the electrical, pneumatic or hydraulic pressure, concave and convex tip deflection, medication delivery and suction functions.

In some embodiments, the device may include, in addition to a channel for pneumatic or hydraulic pressure running the length of the guidance stylet, a channel for delivering medication from the handpiece, exiting as a spray from the internal tip of the guidance stylet.

In some embodiments, the device may include an additional hollow channel in the guidance stylet that runs from the internal tip to the handpiece and may allow for both receiving a guidewire placed percutaneously from below the vocal cords for retrograde endotracheal intubation and for suction of material from the area at the internal tip of the guidance stylet.

In some embodiments, the base of the handpiece may have a tubular connector which may snugly overlie the tubular connector on an external end of the endotracheal breathing tube and may allow for guidance of the endotracheal breathing tube by moving the handpiece.

Embodiments may include a device for guiding placement of an endotracheal breathing tube using a pneumatic or hydraulic handpiece (e.g., via fluid pressure). Embodiments may improve intubation procedures by providing for success on a first attempt to intubate a patient and improving the ease of intubation, such as during laryngoscopy and placement of an endotracheal breathing tube. Embodiments may be used alone, without other airway devices, or may be used in combination with many other airway devices, such as oral airways or laryngeal mask airways. Embodiments provide for ease of intubation by allowing for adjustment of fluid pressure in embodiments via tightening and/or relaxing pressure on a compressible handpiece of some embodiments (e.g., via an operator's grip on a pneumatic or hydraulic handpiece). For example, an operator may increase or decrease an angulation of some embodiments away from a resting shape (e.g., a straight, linear shape) by increasing or decreasing pressure on a compressible handpiece. Different magnitudes of fluid pressure (e.g., pneumatic or hydraulic) may increase or decrease a degree of angulation and may also engage or disengage locations of flexion in some embodiments. Such real-time adjustment of a stylet in various embodiments, which may be placed within a lumen of an endotracheal breathing tube, may facilitate successful placement of the endotracheal breathing tube between vocal cords of a patient and into a trachea of the patient on a first attempt, with minimal to no difficulties and/or or errors for an operator (e.g., a medical practitioner).

Embodiments may include at least two conjoined components, for example a handpiece and a stylet. The handpiece may be held in the right hand and may include means whereby a medical professional manually controls placement of an endotracheal breathing tube. A length of the stylet may be approximately (e.g., substantially equal to) a length of an endotracheal breathing tube. Before an intubation procedure, the stylet may be inserted into a lumen of the endotracheal breathing tube and may be advanced from an external end, which may be connected to a ventilator, down to an internal end which may be inserted in a trachea of a patient.

In some embodiments, a stylet may be composed of a rigid or a semi-rigid polymer which, in a resting state (e.g., unflexed, not subject to bending and/or torsion, and/or the like) forms a straight and/or linear component. The stylet composed of the rigid or semi-rigid polymer may be deformable into a curved shape (e.g., via flexion, bending, and or torsion forces, and/or the like) using pressure (e.g., fluid pressure). In some embodiments, a stylet may include at least one hollow channel (e.g., internal channels) that continuously run a length of the stylet. For example, a stylet may include a channel to transmit fluid pressure (e.g., pneumatic pressure or hydraulic pressure), a channel for suction of airway material, and/or a channel for medication administration. A channel for medication administration may terminate in a baffle so that liquid medication may be transformed into and administered as a spray as the liquid medication exits the stylet.

A distal portion of the stylet may include a plurality of flat empty pockets that may be composed of nylon or a similar non-distensible, non-porous and/or airtight polymer. Each pocket may include an aperture in a wall of a channel to transmit fluid pressure (e.g., pneumatic pressure or hydraulic pressure), the aperture leading into a pocket to allow the fluid to flow into the pocket and allow the pressure to transform each pocket into a spherical shape (e.g., a more spherical shape, a spherical shape, or a substantially spherical shape), thereby causing the stylet to adopt a curved shape, from the stylet's initial straight, linear shape.

The pockets may include an elastic polymer coating over the pockets which may restrict the pocket from filling with fluid (e.g., air, water, another gas or liquid, and/or the like) below a certain pneumatic or hydraulic pressure threshold. The elastic polymer coating may include a plurality of occlusion pressures for the pockets. Thus, in some embodiments, increasing compression on a handpiece may cause inflation of the pockets which may result in increasing a convexity (e.g., a degree of curve) of the stylet where inflated pockets may be located. In some embodiments, increasing compression on a handpiece (e.g., as pneumatic or hydraulic pressure increases), additional pockets of the plurality of pockets may inflate (e.g., locations of pockets based on data from radiologic studies of human airway anatomy) causing additional areas of the stylet to contribute to the degree of curvature.

In some embodiments, at an internal tip of the stylet, the stylet may include a LED and a video camera. One or more wires may run up to and may be connected with a power source located at or within the handpiece. The video camera may collect video data of vocal cords of a patient and the video data may be transmitted to and displayed via a display device (including at least one display screen). The display device may be arranged on the handpiece or another part of the stylet, or in some embodiments the display device may be separate and/or remote from the stylet and/or handpiece. For example, the display device may display a video laryngoscope if a video laryngoscope is being used in a patient. In some embodiments, a device (e.g., stylet and handpiece) may include electronic components to enable video transmission from the camera to another display device (e.g., client device, mobile device, and/or the like) for remote viewing.

In some embodiments, at least one of the channels may be connected to a suction device (e.g., a suction channel) so that when a suction port on the handpiece is under suction, a suction port at the internal tip of the stylet will aspirate airway material. The suction channel may include a wide opening at the internal tip of the stylet so that the suction channel may also be used to receive a guidewire or catheter inserted into the airway percutaneously below vocal cords of a patient. The guidewire may be used to place an endotracheal breathing tube in a trachea to complete a retrograde endotracheal intubation.

In some embodiments, at the internal tip of the stylet, the stylet may include a polymer fiber from a convex side extending from the internal tip of the stylet up to the handpiece. Inducing tension on the polymer fiber (e.g., by applying pressure to a tension control unit located in the handpiece) may provide convex angulation of the tip of the stylet. This convex angulation may be opposite to angulation induced in the remainder of the endotracheal breathing tube by the stylet. In this way, the convex angulation may be useful when entering a glottic opening and when trying to perform a one-sided endobronchial intubation.

Embodiments may be used during laryngoscopy or for any other means of performing endotracheal intubation. For example, an awake intubation with or without the use of any additional instrumentation or airway guides or devices is possible with embodiments described herein.

Embodiments may be useful for receiving a wire in a pharynx of a patient when performing a retrograde endotracheal intubation. In a retrograde tracheal intubation, a needle may be inserted percutaneously through skin of the patient in the front of the patient's neck and deeper through a cricothyroid membrane into the patient's trachea just caudal to the patient's vocal cords. A needle may be angled with the tip of the stylet in the cephalad direction and a wire may be blindly inserted through a lumen of the needle. Unless the wire becomes lodged in friable mucosal tissue of the pharynx, the wire may ascend through the pharynx and exit from the patient's mouth or nose as the wire is blindly advanced. Both ends of the wire may be secured (e.g., one end protruding from the skin in the front of the neck and the other end exiting the mouth), and the wire exiting the mouth may be threaded through a lumen of an endotracheal breathing tube. The endotracheal breathing tube may then be advanced over the wire until the internal tip of the endotracheal tube is resting in the trachea of the patient. Using embodiments in such a way may ensure that the wire, which may be blindly advanced through the needle in the front of the patient's neck, is not lost or entrapped in the friable mucosal tissue of the patient's pharynx or that the wire is unretrievable because of bleeding or gastric contents in the patient's throat.

Some embodiments may be used for performing nasal intubations. Nasal intubations may involve inserting an endotracheal breathing tube through a nostril until the tip of the endotracheal breathing tube is resting in the posterior pharynx of a patient. After hypnotic and muscle relaxing medications are administered, laryngoscopy may be performed and a tip of the endotracheal breathing tube may be grasped with forceps and directed further between the vocal cords into the trachea. Sometimes, no laryngoscopy is performed and no muscle relaxant medications are used. In these patients, anesthetic medicines may be administered and in a spontaneously breathing patient the endotracheal breathing tube may be inserted through the nostril and advanced carefully, but blindly and repeatedly, until exhalation is detected in the endotracheal breathing tube. Alternatively, the endotracheal breathing tube may be inserted through the nostril until the tip of the endotracheal breathing tube is in the posterior pharynx of the patient and a flexible video bronchoscope may be inserted through a lumen of the endotracheal breathing tube. The flexible video bronchoscope may be directed into the trachea of the patient and the endotracheal breathing tube is then advanced, using the bronchoscope as a guide, into the trachea of the patient.

In some embodiments, the handpiece and the stylet may be physically separate components. Where the handpiece and the stylet are physically separate components, the handpiece and/or the stylet may be configured to connect in such a way that the pneumatic or hydraulic pressure generated by compressing the handpiece may be reliably transmitted to the stylet and to a plurality of pockets in the internal tip of the stylet, the electrical connections between the LED and video camera may be maintained, suction is delivered from the handpiece to the internal tip of the stylet, concave deflection of the tip of the stylet may still be induced by compressing the compressible handpiece (e.g., when the compressible handpiece and stylet are connected), while convex deflection of the tip of the stylet may be induced by pressure applied to the control lever on the compressible handpiece (e.g., depressing the control lever or control button), and the medication which is delivered from the handpiece reaches a baffle at the distal end of the stylet.

The handpiece may include a medication dispersal section that may cause medication in a medication dispersal channel (e.g., a medication dispersal section) to be subject to compression. In some embodiments, any medication could be used. For example, embodiments may use a local anesthetic. Tubing from the medication dispersal section under compression may be occluded until the medication dispersal section is opened.

These and other features and characteristics of the present disclosure, as well as the methods of operation and functions of the related elements of structures and the combination of parts and economies of manufacture, will become more apparent upon consideration of the following description and the appended claims with reference to the accompanying drawings and appendix, all of which form a part of this specification, wherein like reference numerals designate corresponding parts in the various figures. It is to be expressly understood, however, that the drawings and appendix are for the purpose of illustration and description only and are not intended as a definition of the limits of the disclosed subject matter.

### BRIEF DESCRIPTION OF THE DRAWINGS

Additional advantages and details are explained in greater detail below with reference to embodiments that are illustrated in the accompanying schematic figures, in which:

FIG. 1 shows a side view of a left hand holding a laryngoscope as it compresses a tongue of a patient and a right hand holding an exemplary stylet in combination with an endotracheal breathing tube overlying the stylet according to embodiments as disclosed herein;

FIG. 2 shows a side view of an exemplary compressible handpiece according to embodiments as disclosed herein;

FIG. 3 shows a side view of an exemplary compressible handpiece including a screen of a display device according to embodiments as disclosed herein;

FIG. 4 shows a cross section view of a middle portion of an exemplary stylet between a handpiece and a curvable section including a plurality of pockets according to embodiments as disclosed herein;

FIG. 5 shows a side view of a curvable section of an exemplary stylet including a tip with a plurality of pockets deflated according to embodiments as disclosed herein;

FIG. 6 shows a front view of a curvable section of an exemplary stylet including a tip with a plurality of pockets deflated according to embodiments as disclosed herein;

FIG. 7 shows a side view of a curvable section of an exemplary stylet including a tip with a plurality of pockets inflated according to embodiments as disclosed herein;

FIG. 8 shows an end view of an internal end of an exemplary stylet according to embodiments as disclosed herein; and

FIG. 9 shows a side view of an exemplary stylet receiving a guidewire for performing a retrograde endotracheal intubation according to embodiments as disclosed herein.

### DETAILED DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the embodiments as they are oriented in the drawing figures. However, it is to be understood that the embodiments may assume various alternative variations and step sequences, except where expressly specified to the contrary. It is also to be understood that the specific devices and processes illustrated in the attached drawings and appendix, and described in the following specification, are simply exemplary embodiments of the disclosed subject matter. Hence, specific dimensions and other physical characteristics related to the embodiments disclosed herein are not to be considered as limiting.

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, and/or the like) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

Embodiments may change a curve of an endotracheal breathing tube while the endotracheal breathing tube is being manually positioned (e.g., by an operator), thereby facilitating accurate placement of the endotracheal breathing tube on a first attempt. Such embodiments may also reduce or eliminate errors in placement and/or positioning of endotracheal breathing tubes by providing an operator with full control of guidance of the endotracheal breathing tube via a stylet inserted into the endotracheal breathing tube. By adjusting compression on a compressible handpiece (e.g., via a grip of a hand of the operator), the operator may precisely alter a degree of curvature of a tip of the endotracheal breathing tube such that the endotracheal breathing tube can be safely and accurately guided and positioned within a patient. A stylet may be inserted into a lumen of the endotracheal breathing tube to control the curvature of the endotracheal breathing tube and/or cause the endotracheal breathing tube to curve and/or deflect. The stylet may include pockets (e.g., airtight pockets) in a continuous channel of the stylet extending to an internal tip of the stylet. For example, the pockets may be attached to an external wall of the continuous channel, each pocket having an aperture between the continuous channel and the pocket to allow fluid to flow from the continuous channel into the pocket. The pockets may be adjustably inflated with air or other fluid by an operator compressing a compressible handpiece. Graded compression of the handpiece (e.g., via a grip of a hand of an operator) may cause and/or transmit fluid pressure in the continuous channel of the stylet and thereby may cause inflation of the pockets in the stylet. The inflation of the pockets in the stylet may cause the stylet to change from having a straight shape (e.g., linear) to having a curved shape (e.g., nonlinear). The change in shape of the stylet may thereby cause the endotracheal breathing tube to conform to and/or retain the curved shape of the stylet with the stylet inserted in the lumen of the endotracheal breathing tube.

Embodiments may be suited for intubation of patients with a possible unstable neck or cervical spine fracture where a forceful laryngoscopy may cause a permanent cervical spinal cord injury. In these patients, minimal force required to achieve visualization of the vocal cords may be used to minimize the likelihood of causing mechanical injury to the spinal cord. Because a pathway from the open mouth to the vocal cords is not straight in these patients, the ability of the embodiments to create a graded curve in real time to suit the anatomy of the patient requiring endotracheal intubation can obviate the need for repeated laryngoscopy and reshaping of the stylet for patients with an unusual anatomy. The endotracheal breathing tube extends from outside the mouth down to and inside the cranial end of the trachea.

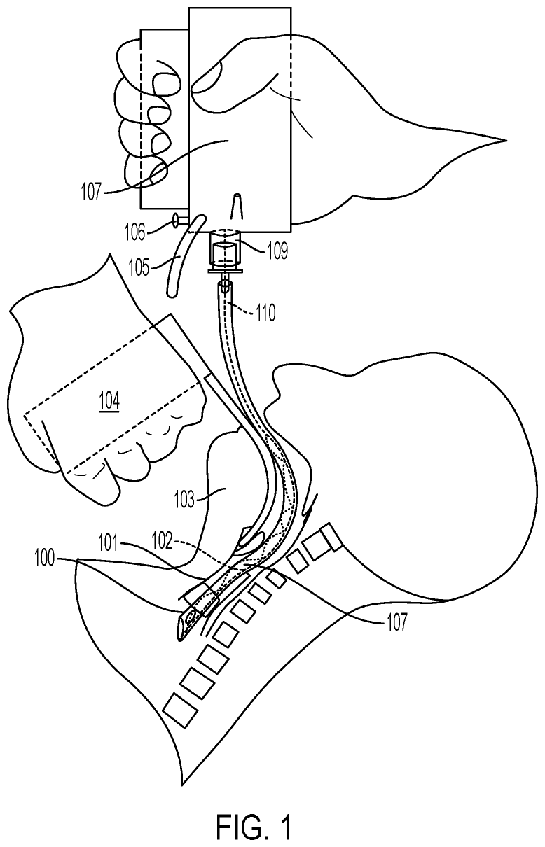

FIG. 1 shows a side view of a left hand holding a laryngoscope handle 104 as it compresses a tongue 103 of a patient and a right hand holding an exemplary device 107 including a stylet 110 in combination with an endotracheal breathing tube 100 overlying a stylet 110 being used during laryngoscopy and placement of the endotracheal breathing tube 100 in the cranial end of the trachea 101 just caudal to the vocal cords 102. The endotracheal breathing tube 100 may be any common commercially available “off the shelf” endotracheal breathing tube without need for any modification. The appropriate size endotracheal breathing tube for the size of the patient may be used, with the appropriate size stylet that corresponds to the size of the endotracheal breathing tube. A left hand may hold the laryngoscope handle 104, and a right hand may hold a compressible handpiece 107.

A stylet 110 may include materials such as, but not limited to, polymeric materials, metallic materials, and/or mixtures thereof. In some embodiments, the stylet 110 may include polymeric materials. The stylet 110 may, for example, include a material that is both sufficiently rigid to be directional (e.g., linear) and/or sufficiently deformable to deflect (e.g., adopt a curve shape) in response to fluid pressure within the stylet 110 and/or within pockets arranged on the stylet 110. The stylet 110 may have a proximal end and a distal end (e.g., a stylet tip).

A stylet 110 may be coupled to (e.g., attached, and/or the like) the compressible handpiece at a proximal end of the stylet 110. A tip of the stylet 110 (e.g., a distal end of the stylet 110, opposite the proximal end of the stylet 110) may be deflected (e.g., forming a curved shape, bending in a concave direction, and/or the like) based on inflation of a plurality of pockets 111 arranged on the stylet 110. A user (e.g., a medical practitioner) may adjust compression on the compressible handpiece 107 by adjusting, for example, a grip strength on the compressible handpiece 107 based upon feedback from both the resistance to compression provided by the compressible handpiece 107 as well as by the visual confirmation of adequacy or inadequacy of a degree of curvature of the stylet 110 that compression has created. Such feedback may allow for increasing or decreasing an amount (or degree) of curvature of the stylet 110 and thus increasing or decreasing an amount of curvature of the endotracheal breathing tube 100 (which may conform to a shape of the stylet 110) while performing a laryngoscopy, instead of interrupting the laryngoscopy in order to accommodate for anatomy of a current patient by manually reshaping the stylet 110 for the endotracheal breathing tube 100 and then repeating the laryngoscopy.

As shown in FIG. 1, the device may include an attachment for suction 108 which may provide an open channel to a tip of the stylet 110 to make it possible to aspirate fluids or particulate material which could be obstructing the view of the vocal cords 102 of a patient as well as providing a channel for receiving a guidewire (e.g., guidewire 901) for performing a retrograde intubation. The internal end of the suction channel may have a relatively wide opening to make it easier to receive a wire when performing a retrograde intubation. Note also there may not be a gap between the stylet 110 and a tip of the endotracheal breathing tube 100. At least one inflated pocket at the internal tip of the stylet 110 may obliterate any gap between the stylet 110 and the endotracheal breathing tube 100. Any gaps between a guidewire and the tip of the endotracheal breathing tube 100 may have been hypothesized to allow tissue from the epiglottis or arytenoids to obstruct the advancement of the endotracheal breathing tube 100 into the trachea. This placement failure when trying to advance the endotracheal breathing tube 100 over a guidewire is unlikely when using various embodiments because there is no gap.

The device may include a control unit 106 for medication delivery as a spray from the tip of the stylet 110. In some embodiments, control unit 106 may be a control button or a control lever (e.g., a button or trigger) to control and/or cause medication delivery through a medication delivery channel within the stylet 110. Medication may be stored under pressure in the compressible handpiece 107 (e.g., in a medication compartment). Episodic delivery of the medication may be controlled by depressing control unit 106 on the compressible handpiece 107. It may be demonstrated that spray of local anesthetic may numb the airway tissue and may reduce the hemodynamic response to intubation.

The device may include a control lever 105 which may be attached and/or coupled to a fiber (e.g., fiber 400) that runs the length of the stylet 110. The fiber may be attached to a convex side of an internal tip of the stylet 110. Tension applied to the fiber may cause deflection of the internal tip of the stylet 110 in a convex direction. The control lever 105 may be actuated by compression via a finger of a user (e.g., a medical practitioner). The control lever 105 may be useful in directing the tip of the stylet 110 and thereby directing the tip of the endotracheal breathing tube 100 between the vocal cords 102 of a patient and in performing a left or right endobronchial intubation.

The device may include a collar 109 on a base of the compressible handpiece 107 that snugly slides over the plastic connector (e.g., a compression fit) of the endotracheal breathing tube 100. The snug connection between the compressible handpiece 107 and the endotracheal breathing tube 100 may allow an operator to manipulate the endotracheal breathing tube 100 while holding only the compressible handpiece 107 of the stylet 110. The snug connection may be separated and the stylet 110 may be withdrawn after the endotracheal breathing tube 100 is correctly positioned in the trachea 101 of a patient so that the ventilator tubing can be connected to the endotracheal breathing tube 100.

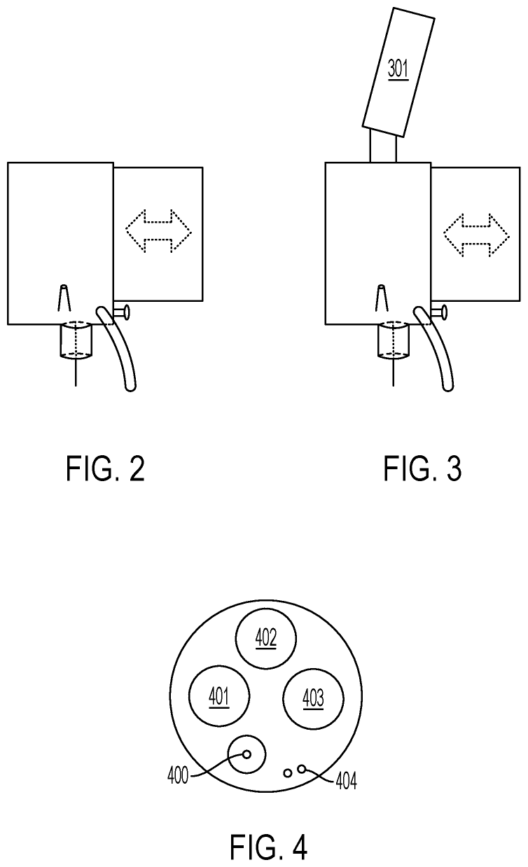

FIG. 2 shows a side view of one embodiment of the compressible handpiece 107. In some embodiments, two sliding components may be squeezed together to build pressure in a fluid (e.g., a gas such as air or a liquid such as water, and/or the like) contained within the compressible handpiece 107. The fluid, in response to an increase in compression pressure on the compressible handpiece 107, may increase pressure down the fluid channel in the stylet 110, out the apertures 501, and into pockets 111 at the internal end of the stylet 110. When the pressure increases in the pockets, the pockets expand, forcing both the free wall to swell out in the middle of the pocket and the other wall, adherent to the stylet 110, to also swell out in a middle of the pockets. The curving of the wall adherent to the stylet 110 may cause the stylet 110 to deflect and/or curve at the location and may force the overlying endotracheal tube to also form the deflection and/or curve at the location. Without fluid pressure transmitted to inside the pockets, the stylet 110 may maintain or return to a straight or linear shape or configuration. Visual feedback of an amount (e.g., degree) of curvature created in the endotracheal breathing tube 100 by compression of the compressible handpiece 107 as well as tactile feedback of resistance to compression may enhance a medical professional's ability to guide the endotracheal breathing tube 100 between the vocal cords 102 of a patient and into the trachea 101 of the patient on the first attempt.

FIG. 3 shows a side view of an embodiment of the compressible handpiece 107. In some embodiments, the device and/or compressible handpiece 107 may include a screen of a display device 301. The screen may be arranged on the compressible handpiece 107 as shown in FIG. 3. The screen of the display device 301 may display a view from a camera arranged in the internal tip of the stylet 110, along with a LED light. The LED and camera in the internal tip of the stylet 110 may show an operator where a tip of the endotracheal breathing tube 100 is positioned relative to anatomic structures of a patient, such as the mouth, pharynx, and larynx. The device and/or compressible handpiece 107 may include a connection between the display device (e.g., the screen of the display device) and the video camera for communication of data. For example, the device may include wires 405 running between the video camera and the screen of the display device arranged on the compressible handpiece 107. The compressible handpiece 107 may include a power supply arranged on the compressible handpiece 107 (e.g., an alternating current (AC) adapter, batteries, and/or the like), and in some embodiments the device may include one or more electronic devices to wirelessly transmit information to a remote display device for remote viewing of the video data at a display device that is separate from the device and/or compressible handpiece 107 at a remote location.

FIG. 4 shows a cross section of an exemplary stylet (e.g., stylet 110) between the handpiece and the curvable section with the plurality of pockets. The stylet may include one or more channels, any one or more channels such as channel for fluid under pressure 401, channel for medication under pressure 402, and/or channel for suction 403. The one or more channels may be continuous from the compressible handpiece 107 to the internal tip end of the stylet which includes the plurality of pockets. Also included in some embodiments is a channel for a fiber 400 which connects the internal stylet tip convex side to the finger control lever 105 in the handpiece as well as, in some embodiments, the wires for power and/or communication for the display device and digital information (e.g., data). The channel for the fiber 400 may rest within the lumen of the endotracheal breathing tube 100. The stylet 110 may also include LEDs 404.

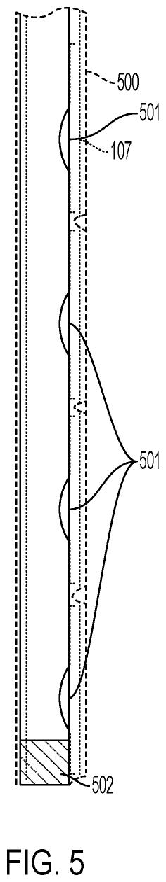

FIG. 5 shows a side view of a curvable section of the internal tip of the stylet. The number of apertures 501 and pockets 111 and the distances between them may vary depending upon a size of the patient and/or a configuration of the device. Overlying all the pockets, like a sock overlying a foot, may be a polymeric elastic material 500 that will not allow the pocket to inflate until the pressure exceeds a pressure threshold (e.g., an opening pressure, aperture pressure, and/or the like), for each pocket. Locations of apertures 501 and pockets may be based upon measurements performed on computerized tomography (CT) scans of airways in adults and children. By increasing the fluid pressure in the stylet, the operator may control some variables. An operator may use embodiments to control an amount of pressure and a degree of angulation at the pockets which may open above the pressure threshold. An operator may use embodiments to control a number of pockets (e.g., pockets at pivot points) which may be inflated (higher pressures exceed more pressure thresholds of pockets and thus inflate more pockets) to create angulation in the stylet. Both the degree of angulation for pockets which may open above the pressure threshold and the number of pockets which may be inflated increase as fluid pressure in the stylet increase as a result of compression on the compressible handpiece 107 (e.g., via grip strength). The tip of the stylet 502 may include the medication spray port, the video camera and light (e.g., LED), and the suction port (e.g., for channel for suction 403) located in and/or at the tip of the stylet 502.

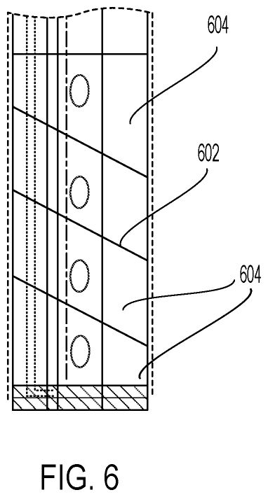

FIG. 6 shows a front view of a curvable section of the internal tip of the stylet. In FIG. 6, the angled divisions 602 separating the pockets 604 may be more apparent. Because of the angled divisions 602 having angulation dividing the pockets 604, the distention of a first pocket may initiate a small amount of initial curvature in an adjacent pocket. The angled divisions 602 and angulation of the divisions between pockets may ensure that all of the pockets may contribute to and/or induce the concave curvature of the stylet 110, instead of alternating between convex and concave curvature of stylet 110. In this way, the stylet 110 may be curved and/or deflected in a same direction (e.g., in a concave direction) by inflation of the pockets 604.

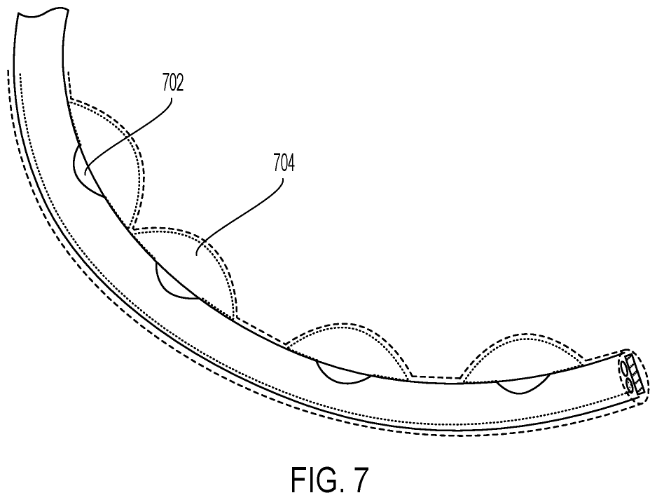

FIG. 7 shows a side view of a curvable section of the stylet (e.g., stylet 110, stylet of FIG. 4) tip having apertures 702 and with pockets 704 inflated. The stylet tip is curved because of the increased tension on the pocket material due to the fluid pressure (e.g., such that the stylet tip creates a graded curve). In FIG. 7, all of the pockets 704 are inflated due to a higher pressure condition in the pockets, as a result of higher fluid pressure in the stylet from compression on the compressible handpiece 107. Each pocket 704 is inflated in a same direction as the other pockets with respect to the stylet to create the graded curve, such that the graded curve of the stylet continues in the same direction (e.g., a concave direction) and so that none of the pockets 704 are working against other pockets 704 to cause the stylet to curve in an opposite direction (e.g., to cause the stylet to return to a straight, linear shape, or curve in a convex direction).

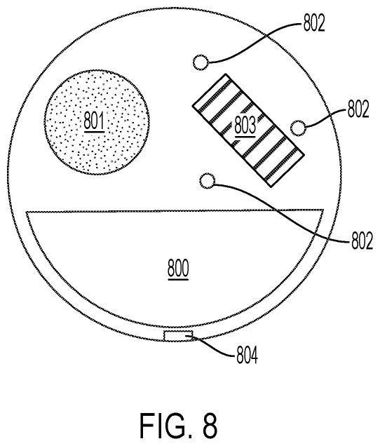

FIG. 8 shows an end view of the internal end of the stylet (e.g., stylet 110, stylet of FIG. 4). An enlarged opening 800 of the channel for suction is shown in FIG. 8. The channel for suction may be the same as or similar to channel for suction 403. The enlarged opening 800 for the channel for suction may be larger at the tip than over some of the length of the channel for suction to facilitate receiving a guide wire into the channel for suction when performing a retrograde endotracheal intubation. In FIG. 8, also shown in a baffle for a medication delivery channel 801 (e.g., the same as or similar to channel for medication under pressure 402), illumination LEDs 802, video lens 803 (e.g., for a video camera), and an attachment 804 for the fiber 400 that may create convex deflection of the tip.

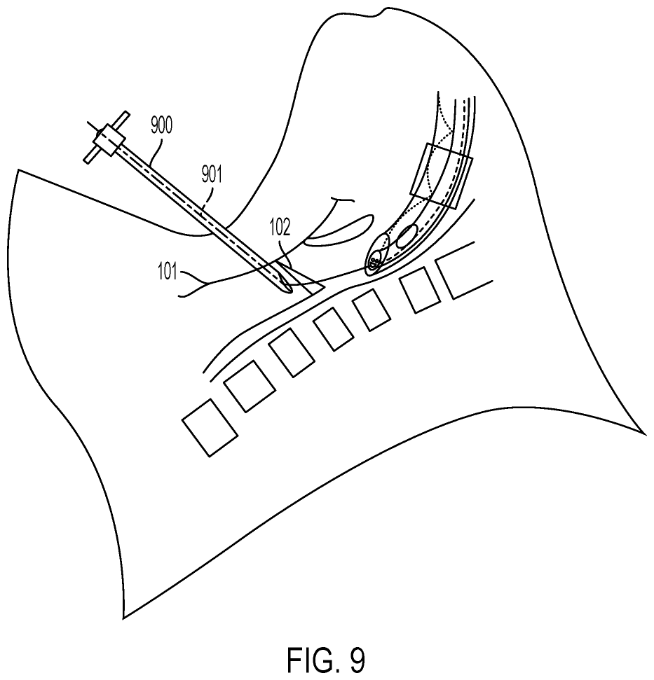

FIG. 9 shows a side view of the internal tip of a stylet (e.g., stylet 110, stylet of FIG. 4), positioned cephalad to vocal cords 102 of a patient, receiving a guidewire 901 in the channel for suction (e.g., channel for suction 403, channel for suction with enlarged opening 800) as would be the situation when performing a retrograde intubation. Some embodiments, in combination with an overlying endotracheal breathing tube 100, may be guided close to a correct position in the pharynx. Numbing medicine may be infiltrated into skin of the patient and/or underlying deeper tissues in the front of the neck. A needle 900 may be inserted through numb skin of the patient and then further through the deeper tissues and slightly further into the trachea 101 of the patient. A guidewire 901 may be inserted through the needle 900 and directed in a cephalad direction through the vocal cords 102 and in the direction of the mouth where it is captured and used as a guide inside an endotracheal breathing tube 100. Holding tension on both a mouth end and a neck end of the guidewire 901, a medical professional may advance the endotracheal breathing tube 100 into the trachea 101 of the patient. This retrograde intubation technique may be used in patients where it may be challenging to correctly place the endotracheal breathing tube 100 because of congenital or acquired variation (e.g., tumor, abscess, inability to open the mouth, and/or the like).

In FIG. 9, the guidewire may enter an enlarged channel for suction of the stylet and may be advanced up the suction channel until the guidewire 901 exits the stylet and can be grasped so that the stylet in combination with the endotracheal breathing tube 100 may be advanced to a desired position in the trachea 101 of the patient. By visualizing the guidewire 901 passing through the vocal cords 102 and into the enlarged suction channel of the stylet, the medical professional may avoid risk of the guidewire 901 tip piercing and/or getting trapped in friable mucosal tissue of the patient while the guidewire 901 is blindly advanced through the needle 900 in the front of the neck of the patient.

Although embodiments have been described in detail for the purpose of illustration, it is to be understood that such detail is solely for that purpose and that the disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect.

What is claimed is:

1. A device for positioning an endotracheal breathing tube, comprising:
a compressible handpiece for producing fluid pressure, wherein the fluid pressure is produced in a graded manner based on an operator compressing the compressible handpiece;
and a stylet linearly extending from a first end of the stylet to a tip of the stylet, the first end of the stylet coupled to the compressible handpiece, the stylet configured to be inserted into a lumen of an endotracheal breathing tube and deflect into a curved shape when the compressible handpiece is compressed to produce fluid pressure within the stylet.

2. The device of claim 1, wherein the stylet is configured to maintain a linear shape and return to the linear shape when the compressible handpiece is not compressed such that no fluid pressure is produced within the stylet.

3. The device of claim 1, further comprising a first continuous channel arranged within an interior of the stylet extending from the first end of the stylet to the tip of the stylet, the first continuous channel including a plurality of airtight pockets including apertures for fluid to enter the airtight pockets, the airtight pockets being distributed along the first continuous channel of the stylet and configured to inflate when the compressible handpiece is compressed to produce fluid pressure within the first continuous channel of the stylet and thereby cause fluid to enter the airtight pockets through the apertures of the airtight pockets.

4. The device of claim 3, wherein the airtight pockets are attached to an external wall of the first continuous channel of the stylet, the airtight pockets configured to cause the stylet to deflect when at least one of the airtight pockets are inflated.

5. The device of claim 1 in combination with an endotracheal breathing tube including a lumen, the stylet being inserted into the lumen of the endotracheal breathing tube and the stylet having a length substantially equal to the length of the endotracheal breathing tube;
wherein when the compressible handpiece is compressed to produce fluid pressure, the fluid pressure will cause the stylet to form a graded curve, thereby causing the endotracheal breathing tube to curve based on the stylet deflecting into a curved shape when the compressible handpiece is compressed to produce fluid pressure within the stylet.

6. The device of claim 3, wherein the airtight pockets are arranged along the continuous channel such that the stylet is configured to deflect and thereby cause an endotracheal breathing tube to achieve a curved shape in conformance with the stylet, the airtight pockets arranged in the continuous channel according to any one or more of:
a degree of angulation induced at each pocket;
a number and location of the airtight pockets used to create the degree of angulation; and/or
a sequence of inflation of the airtight pockets and the degree of angulation as the compression is increased on the compressible handpiece to exceed an opening pressure.

7. The device of claim 1, wherein the compressible handpiece is configured to generate immediate tactile and visual feedback based on a relationship between compression of the compressible handpiece and a degree of curvature of the stylet.

8. The device of claim 1, further comprising:
a fiber extending from a convex surface of the tip of the stylet to a tension control unit attached to the compressible handpiece, the fiber coupled with the tension control unit, wherein the fiber and tension control unit are configured to permit deflection of the tip of the stylet in the convex direction when tension is applied to said fiber.

9. The device of claim 1, in combination with:
a control unit;
at least one electrical power source; and
a light emitting diode (LED) and a video camera electrically coupled to the at least one electrical power source, wherein the LED and the video camera are arranged internal to the stylet at the tip of the stylet to;
and a display device electrically coupled to the at least one electrical power source.

10. The device of claim 1, wherein the compressible handpiece and the stylet are independent components that are configured to be coupled and decoupled while preserving any one or more of:
electrical function, fluid pressure function causing concave stylet deflection, concave tip deflection, convex tip deflection, medication delivery and/or suction function.

11. The device of claim 3, further comprising:
a second continuous channel arranged within the interior of the stylet extending from the first end of the stylet to the tip of the stylet, the second continuous channel configured to deliver medication from the compressible handpiece at the first end of the stylet to the tip of the stylet, wherein when the control button for the medication release in the compressible handpiece is depressed, the medication exits the tip of the stylet as a spray.

12. The device of claim 1, further comprising:
a hollow continuous channel arranged within the interior of the stylet extending from the first end of the stylet to the tip of the stylet, the hollow continuous channel configured to receive a guidewire placed percutaneously from below vocal cords of a patient for retrograde endotracheal intubation and for suction of material from an area at the tip of the stylet.

13. The device of claim 1, further comprising a first tubular connector attached to the compressible handpiece, the first tubular connector configured to couple with a tubular connector on an external end of an endotracheal breathing tube to provide for movement of the endotracheal breathing tube by moving the compressible handpiece.

14. A method for positioning an endotracheal breathing tube, comprising:
inserting a stylet into a lumen of an endotracheal breathing tube;
compressing a compressible handpiece coupled to the stylet, wherein compressing the compressible handpiece causes fluid pressure to increase in a first channel within the stylet, thereby causing the stylet to deflect and the endotracheal breathing tube to deflect in conformance with the stylet;
positioning the endotracheal breathing tube in a patient by moving the stylet via the compressible handpiece; and
removing the stylet from the endotracheal breathing tube once the endotracheal breathing tube is positioned in the patient.

15. The method of claim 14, wherein positioning the endotracheal breathing tube includes positioning the endotracheal breathing tube in a trachea of the patient.

16. The method of claim 14, wherein positioning the endotracheal breathing tube includes positioning the endotracheal breathing tube in a posterior pharynx of the patient, the method further comprising:
inserting a needle into a front of a neck of the patient such that the needle is inserted into a trachea of the patient;
inserting a guidewire blindly through the needle such that the guidewire advances in a cephalad direction through vocal cords of the patient producing, with a video camera attached to the stylet and a display device, a visual display of the guidewire in the patient;
retrieving, with a suction channel of the stylet, the guidewire such that the guidewire is advanced up the suction channel of the stylet where the guidewire exits the compressible handpiece; and
advancing the endotracheal breathing tube into the trachea of the patient using the guidewire.

17. A device, comprising:
a stylet including a distal end and a proximal end, wherein the stylet is positioned in an endotracheal tube; and
a handpiece removably coupled to the proximal end of the stylet, wherein the handpiece is compressible and configured to transmit fluid pressure to an internal portion of the stylet,
wherein the stylet is configured to change shape upon transmission of fluid pressure from the handpiece.

18. The device of claim 17, further comprising a camera positioned at the distal end of the stylet.

19. The device of claim 17, wherein the stylet is configured to deflect upon transmission of fluid pressure from the handpiece.
